(19) United States
(12) Patent Application Publication
(10) Pub. No.: US 2022/0241573 A1
(43) Pub. Date: Aug. 4, 2022

(54) DIALYSER CONNECTOR

(71) Applicant: Quanta Dialysis Technologies Limited, Warwickshire (GB)

(72) Inventor: Sam FINCHAM, Warwickshire (GB)

(21) Appl. No.: 17/615,490

(22) PCT Filed: Apr. 21, 2020
(86) PCT No.: PCT/GB2020/050988

§ 371 Date: Nov. 30, 2021

(30) Foreign Application Priority Data
May 31, 2019 (GB) 1907762.7

(51) Int. Cl. A61M 39/10; A61M 1/14; A61M 39/12; F16L 37/098

(52) U.S. Cl. CPC A61M 39/1011; A61M 1/14; A61M 2039/1027; F16L 37/0985; A61M 39/12

(57) ABSTRACT

A dialyser connector comprising a main body and a sliding clip. The main body defines a cavity. The main body has a tube end and a dialyser end, with at least one aperture and a pair of ramps provided in a central portion between the tube end and the dialyser end. The sliding clip has a tube end and a dialyser end connected by a spine running along the top of the sliding clip. The sliding clip has a pair of flexible wings extending in an arc from the spine and a pair of arms extending in an arc from the spine, the pair of arms having at least one projection extending radially inward. The dialyser connector has a locked condition in which the at least one projection extends into the cavity of the main body and an unlocked conditions in which the at least one projection is withdrawn from the cavity of the main body, and wherein the at least one projection is biased to locked condition by action of pair of flexible wings on the pair of ramps.

### TECHNICAL FIELD

The present disclosure relates to a dialyser connector. Particularly, but not exclusively, the disclosure relates to a dialyser connector comprising a main body and a sliding clip. Aspects of the invention relate to a dialyser connector, to a dialysate mixing and pumping cartridge comprising a dialyser connector, to a method of assembling a dialyser connector and to a method of connecting a dialyser connector to a dialyser filter connector port.

### BACKGROUND

A dialysis machine filters a patient's blood of toxins by pumping the blood through a disposable dialyser filter on one side of a semi-permeable membrane and pumping clean dialysate fluid through the disposable dialyser filter on the other side of the semi-permeable membrane. This allows the toxins to move across the semi-permeable membrane into the dialysate fluid and be removed from the blood. The blood and dialysate are pumped to and from the dialyser filter along fluid lines.

To connect the fluid lines to the connector ports of the dialyser filter, two types of connectors are used—one is a screw thread and the other is a push fit. The geometries of both of these types of connector ports are detailed in the standard ISO 8637-1.

Typically, dialysis machines use reusable versions of the push fit connectors while the screw fit connectors are part of the disposable blood tube sets. As the push fit connectors form part of the dialysate circuit, they require sterilization using hot water after each treatment and further chemical treatment on a regularly scheduled basis. As the push fit connectors are designed to be used many hundreds or thousands of times, these connectors are also designed to be robust, and are therefore expensive.

It is known to use a disposable cartridge to produce dialysate fluid and pump this through the dialyser filter. As a result, a disposable push fit connector is also required to fluidically connect the cartridge to the dialyser filter. Disposable push fit connectors are already available and are typically moulded as a single component from a flexible material which conforms around one of the connection ports on a dialyser filter. These connectors have the disadvantage of being very difficult to attach and remove and also may be prone to being pushed off by high pressures, especially after use over time.

This invention relates to a new design of push fit dialyser connector which is inexpensive enough to be disposable but strong enough to be more reliable and convenient for a user.

### SUMMARY OF THE INVENTION

Aspects and embodiments of the invention provide a dialyser connector, a dialysate mixing and pumping cartridge comprising a dialyser connector, a method of assembling a dialyser connector and a method of connecting a dialyser connector to a dialyser filter connector port as claimed in the appended claims.

According to an aspect of the invention, there is provided a dialyser connector comprising a main body and a sliding clip, the main body has a tube end and a dialyser end, the main body defines a cavity, at least one aperture and a pair of ramps, and the sliding clip has a tube end and a dialyser end, the sliding clip defines a spine connecting the tube end and a dialyser end, the sliding clip has a pair of flexible wings extending in an arc from the spine and a pair of arms extending in an arc from the spine, the pair of arms have at least one projection extending radially inward, wherein the dialyser connector has a locked condition in which the at least one projection extends into the cavity of the main body and an unlocked conditions in which the at least one projection is withdrawn from the cavity of the main body, and wherein the at least one projection is biased to the locked condition by resilient abutment of pair of flexible wings on the pair of ramps.

The at least one projection may comprise a pair of projections and the at least one aperture may comprise a pair of apertures.

At least one arm of the pair of arms may have a hook configured to abut a shoulder of the main body to prevent detachment of the sliding clip from the main body when assembled.

The central portion may be bounded toward the tube end by a circumferential flange, preferably the central portion may be partially bounded toward the dialyser end by two arcuate flanges.

The spine may have a depression

The main body may have a projection, preferably wherein the spine has a notch.

The projection may be aligned with the vertical plane and extends from an upper surface of the main body.

The dialyser connector may further comprise a seal, preferably wherein the seal comprises an O-ring seal. Alternatively, the seal may comprise an overmould on the main body.

The dialyser connector may be symmetrical about a centred vertical plane.

The dialyser connector may be single use. The dialyser connector may be plastic.

One of the pair of arms may have a hinged latch. The hinged latch may be connected to the arm by a living hinge. The hinged latch may terminate in a wedge shape and the other of the pair of arms may have a corresponding ledge.

According to another aspect of the invention, there is provided a dialysate mixing and pumping cartridge comprising a dialyser connector as hereinbefore defined.

According to yet another aspect of the invention, there is provided a method of assembling a dialyser connector as hereinbefore defined, the method comprising the steps of: inserting an O-ring seal axially into the main body 30 from the dialyser end;

mounting the sliding clip over the main body; slotting the pair of arms over the central portion with the at least one projection dropping into the at least one aperture with a snap fit connection, wherein the arm comprises a hook which abuts a shoulder of the main body to prevent detachment of the sliding clip from the main body when assembled.

According to a further aspect of the invention, there is provided a method of connecting a dialyser connector to a dialyser filter connector port, the method comprising the steps of providing a dialyser connector as hereinbefore defined, depressing the spine of the sliding clip relative to the main body so as to withdraw the at least one projection from the recess of a dialyser filter connector port, connecting the dialyser connector to the dialyser filter connector port, releasing the spine of the sliding clip relative to the main body so that the dialyser connector automatically assumes a locked condition in which the at least one projection engages a recess of a dialyser filter connector port under the biasing action of the pair of flexible wings on the pair of ramps.

The method may further comprise the step of disconnecting the dialyser connector from the dialyser filter connector port by depressing the spine of the sliding clip relative to the main body so as to withdraw the at least one projection from the recess of a dialyser filter connector port, and withdrawing the dialyser connector from the dialyser filter connector port.

### BRIEF DESCRIPTION OF THE DRAWINGS

One or more embodiments of the invention will now be described, by way of example only, with reference to the accompanying drawings, in which:

FIG. 1 is an exploded schematic view of a dialyser connector according to an aspect of the invention;

FIG. 2 is a schematic view of the dialyser connector of FIG. 1 in a locked condition;

FIG. 3 is a schematic view of the dialyser connector of FIG. 1 in an unlocked condition;

FIG. 4 is a cross-sectional view of the dialyser connector of FIG. 1 in an unlocked condition;

FIG. 5 is a partial section view of the dialyser connector of FIG. 1 in a locked condition; and

FIG. 6 is a schematic representation of the dialyser connector of FIG. 1 in use;

FIG. 7 is an exploded schematic view of a dialyser connector according to a further aspect of the invention;

FIG. 8 is a schematic view of the dialyser connector of FIG. 7 in an unlocked condition;

FIG. 9 is a schematic view of the dialyser connector of FIG. 7 in a locked condition; and

FIG. 10 is a schematic view of the dialyser connector of FIG. 7 in a locked condition.

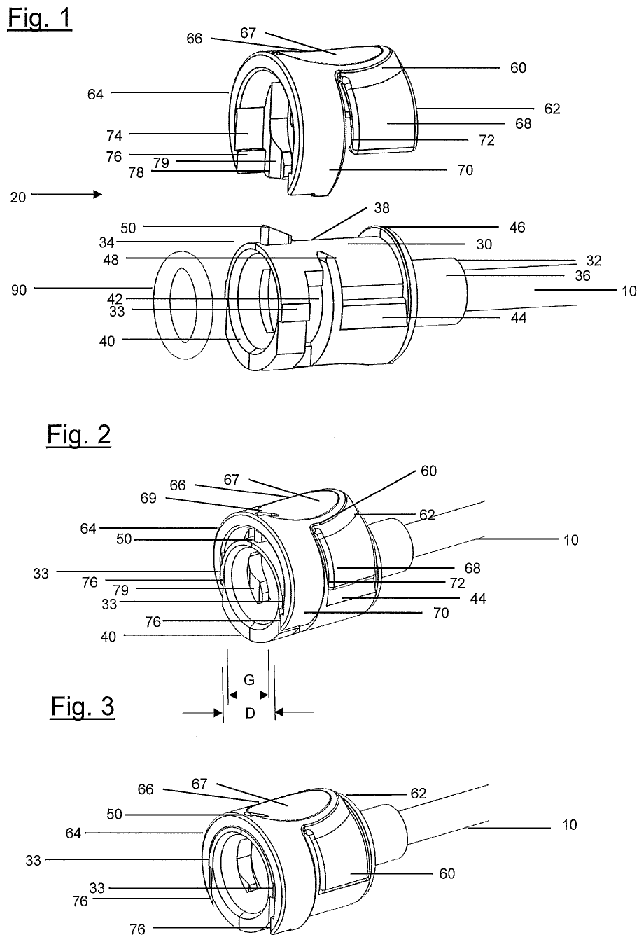

### DETAILED DESCRIPTION

The dialyser connector 20 comprises a main body 30, a sliding clip 60 and an O-ring seal 90.

The dialyser connector 20 has a tube end 22 configured for connection to flexible dialysate tubing 10 and a dialyser end 24, configured for connection to a dialyser filter connector port 110, as will be described in more detail below. Looking end-on, in the orientation shown in all figures, the dialyser connector 20 is symmetrical about a vertical plane passing between the tube end 22 and the dialyser end 24.

The main body 30 is a single piece. The main body 30 defines a cylindrical cavity. The main body 30 has a tube end 32 and a dialyser end 34. Looking end-on, the main body 30 is symmetrical about a vertical plane passing between the tube end 32 and the dialyser end 34.

The tube end 32 and the dialyser end 34 are separated by a central portion 38. The tube end 32 terminates in a tube connector 36. The dialyser end 34 includes a shoulder 33 on each side. The dialyser end 34 terminates in an annulus 40. The annulus 40 is separated from the central portion 38 by two apertures 42. The two apertures 42 being provided on either side of the vertical plane.

The central portion includes a ramp 44 on either side, increasing a wall thickness of the central portion 38. The central portion 38 is bounded toward the tube end 32 by a circumferential flange 46. The central portion 38 is partially bounded toward the dialyser end 34 by two arcuate flanges 48.

A projection 50 extends from an upper surface of annulus 40. The projection is aligned with the vertical plane.

The sliding clip 60 is a single piece. The sliding clip 60 has an arcuate form, having a tube end 62 and a dialyser end 64. Looking end-on, the sliding clip 60 is symmetrical about a vertical plane passing between the tube end 62 and the dialyser end 64.

The tube end 62 and the dialyser end 64 are connected by a spine 66 running along the top of the sliding clip 60. The spine 66 has a depression 67. The spine has a notch 69, centrally aligned and toward the dialyser end 64. The tube end 62 comprises a pair of flexible wings 68 which extend in approximately 90 degree arcs from the spine 66, along either side of the sliding clip 60. The dialyser end 64 comprises a pair of arms 70 which extend in approximately 135 degree arcs from the spine 60, along either side of the sliding clip 60. The pair of flexible wings 68 are separated from the pair of arms 70 by respective cut-outs 72.

A dialyser end portion of an inside surface of each of the pair of arms 70 includes a cut-out 74 which defines a hook 76 adjacent the end of each arc. A tube end portion of the inside surface of each of the pair of arms 70 has a projection 78 adjacent the cut-outs 72. The projections 78 extend radially inward, along a portion of each arc. The projections 78 have a chamfer 79 facing the dialyser end 64.

Assembly

The O-ring seal 90 is inserted axially into the main body 30 from the dialyser end 34, and retained in an annular recess 52 of an inside surface of the main body 30, adjacent the tube end 32.

The dialyser connector 20 is assembled with the sliding clip 60 mounted over the main body 30.

At the dialyser end 34, the pair of arms 70 are slotted over the central portion 38, with the projections 78 dropping into the respective apertures 42. This is a snap fit connection, the pair of arms 70 must flex outwardly when contacting the central portion 38, given that the gap G between the innermost points of the projections 78 is smaller than the distance D on the main body 30 between the apertures 42. Furthermore, each hook 76 of the pair of arms 70 abuts the shoulder 33 of the main body 30 to prevent detachment of sliding clip 60 from the main body 30. At the tube end 32, the pair of flexible wings 68 contact the ramp 44 on either side. The projections 78 impinge radially inwards through the apertures 42, and into the cavity of the main body, such that they are visible when viewing the dialyser connector 20 from the dialyser end 34.

The projection 50 extends through the notch 69 so that the projection is visible on the spine 66 of the sliding clip 60.

Usage

Once assembled, the dialyser connector 20 may be manipulated between a locked condition in which the projections 78 engage a circumferential recess 112 of a dialyser filter connector port 110 and an unlocked conditions in which the projections 78 are withdrawn from the circumferential recess 112 of a dialyser filter connector port 110.

The contact of the ramps 44 on the pair of flexible wings 68 biases the dialyser connector 10 to the locked condition. Thus the projections 78 are biased to engage the circumferential recess 112 of the dialyser filter connector port 110.

Finger pressure upon the depression 67 of the spine 66 of the sliding clip 60 overcomes the biasing force as the pair of flexible wings 68 flex progressively up the inclined plane described by each ramp 44, allowing downward movement of the sliding clip 60 relative to the main body 30. The downward movement withdraws the projections 78 from impinging radially inwards through the apertures 42, and thus out of engagement with the circumferential recess 112 of a dialyser filter connector port 110. This allows removal of the dialyser connector 20 from the dialyser filter connector port 110.

Because of the resilient nature of the flexible wings 68, the dialyser connector 20 returns to the locked condition when the finger pressure is removed as the flexible wings 68 slide down the inclined plane described by each ramp 44, thus returning the flexible wings 68 to an un-flexed condition.

The projection 50 acts as an alignment feature to ensure that the dialyser connector 10 remains aligned vertically as it slides downwards. This also gives a visual indication to a user that the dialyser connector 20 has been fully depressed or that the dialyser connector 20 has fully sprung back to the locked condition.

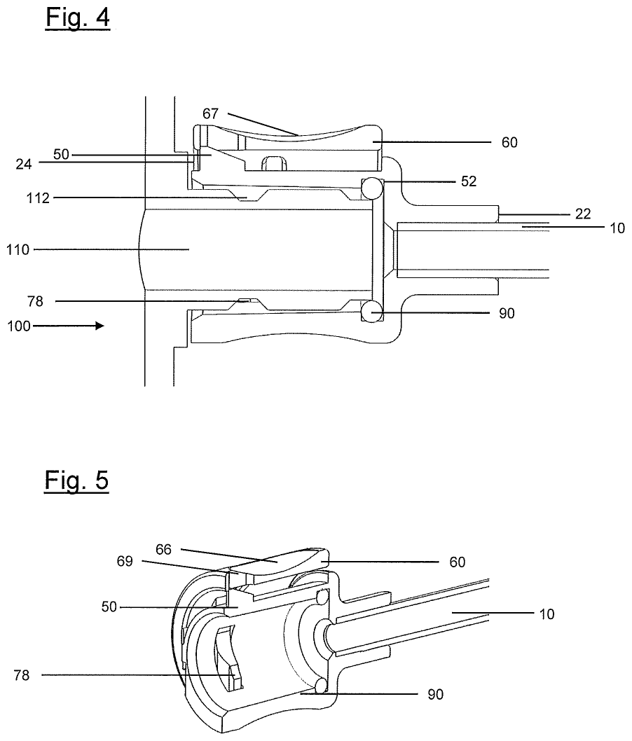

Referring to FIGS. 4 and 5, the O-ring seal 90 retained in an annular recess 52 of an inside surface of the main body 30, and abuts an outer end surface of the dialyser port 110. This provides a fluid tight seal between the dialyser connector 20 and the dialyser 100. The dialyser port 110 could be an inlet or and outlet of the dialyser 100.

The chamfer 79 on the projections 78 forces the sliding clip 60 downwards as it is pushed onto a dialyser port 110. This allows the connector 20 to be attached to a dialyser port 110 without pushing the sliding clip 60 down manually. The right angle on the rear of the projections 78 prevents the connector 20 from being removed without first pushing the sliding clip 60 down. Furthermore, the projection 50 maintains the sliding clip 60 straight as it slides down relative to the main body 30.

The dialyser connector 20 or the sliding clip 60 may be colour-coded to aid differentiation of the inlet and outlet connectors.

In an alternate embodiment, the seal is provided as an overmould on the main body 30. The overmould seal seals against the dialyser in the same manner as the O-ring seal 90.

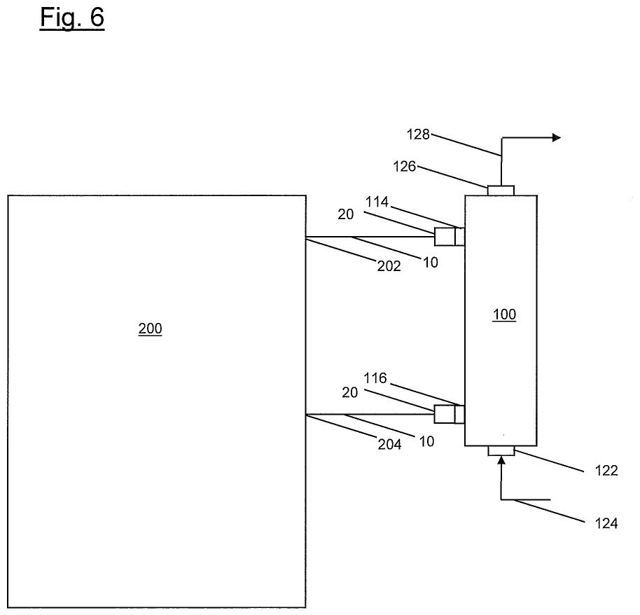

FIG. 6 shows a schematic representation of the dialyser connector 20 in use with a disposable cartridge 200, for example the dialysate mixing and pumping cassette of WO 2010/146344 the entire contents of which are incorporated herein by reference, or the dialysate mixing and pumping cassette of WO 2013/110919 the entire contents of which are incorporated herein by reference.

The disposable cartridge 200 is responsible for pumping and mixing dialysate and has a clean dialysate outlet port 202 and a spent dialysis inlet port 204. These ports 202, 204 are fluidically connected to dialyser 100. Dialyser 100 has a blood inlet port 122 for receiving blood from arterial blood line 124 and blood outlet port 126 for sending blood to venous blood line 128. Dialyser 100 further has dialyser filter connector ports 110, in this case identified by dialysate inlet port 114 and dialysate outlet port 116. Two dialyser connectors 20 are used. One dialyser connector 20 fluidically connects clean dialysate outlet port 202 to the dialysate inlet port 114 using flexible dialysate tubing 10. Another dialyser connector 20 fluidically connects spent dialysate inlet port 204 to the dialysate outlet port 116 using flexible dialysate tubing 10. The flexible dialysate tubing 10 may be made from PVC. The flexible dialysate tubing 10 may be solvent bonded to the tube connector 36. In one embodiment, an outer surface of the PVC dialysate tubing 10 is coated with a mix of butanone and cycolohexanone and pushed into the tube connector 36. The solvent melts the two surfaces together, then evaporates to form a bond.

In an alternate embodiment, the tube end 22 of the dialyser connectors 20 terminate in a cartridge connector (not shown). The cartridge connector connects directly to the disposable cartridge 200, without the need for flexible dialysate tubing 10.

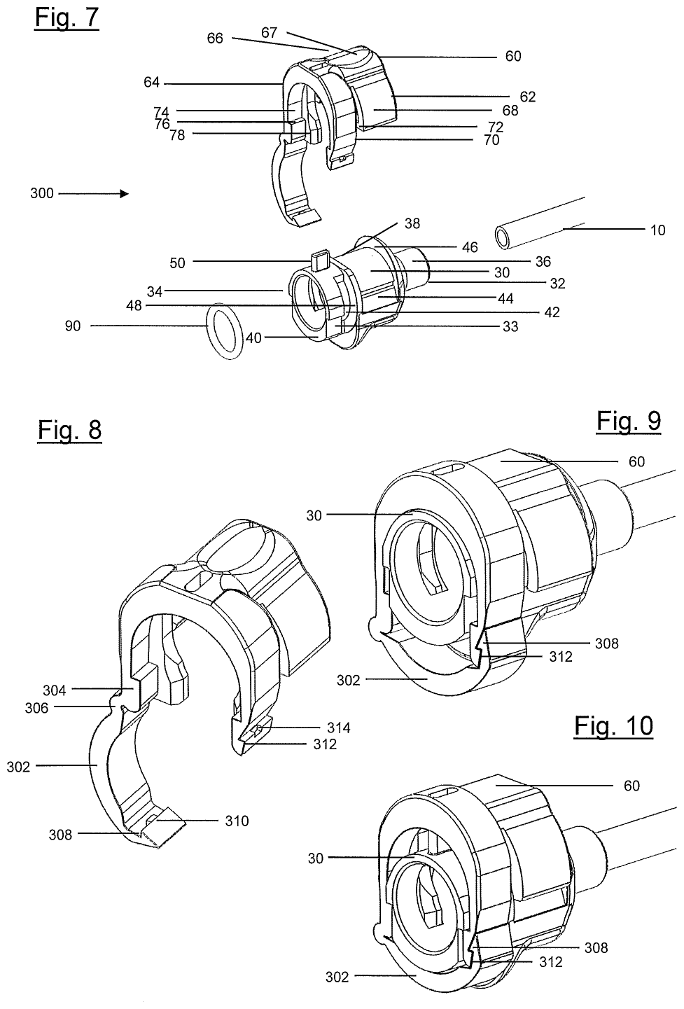

FIGS. 7 to 10 show an alternate embodiment of a dialyser connector generally designated 300. The dialyser connector 300 includes many of the features of dialyser connector 20 described above, only the main differences shall be described in detail.

Similar reference numbers are used to designate similar features as with respect to dialyser connector 20.

In the dialyser connector 300, one of the pair of arms 70 of the dialyser end 64 of the sliding clip 60 comprises a hinged latch 302. The hinged latch 302 extends from an end 304 of one the pair of arms 70 by means of a living hinge 306. The hinged latch 302 terminates in a wedge shape 308. The wedge shape 308 includes a projection 310. The other of the pair of arms 70 terminates in a corresponding ledge 312. The ledge 312 has a corresponding recess 314.

During use, the hinged latch 302 folds up and locks in place by means of the projection 310 engaging with recess 314, as the wedge shape 308 hooks over ledge 312.

The sliding clip 60 is still able to be moved relative to the main body 30, as per the dialyser connector 20 (see FIGS. 9 and 10).

The hinged latch 302 maintains the dialyser connector 300 firmly attached to the dialyser filter connector port 110. Furthermore, the hinged latch 302 prevents the pair of arms 70 from flexing outwards under excess pressure. The projection 310 and corresponding recess 314 provides an alignment feature which prevents the hinged latch 302 from axial movement relative to the ledge 312.

The hinged latch 302 may be released from engagement with the other of the pair of arms 70 by squeezing the pair of arms 70 together whilst stretching the hinged latch 302 to disengage the wedge shape 308 from ledge 312.

### LIST OF REFERENCE NUMBERS

flexible dialysate tubing 10
Dialyser connector 20; 300
tube end 22
dialyser end 24
main body 30
tube end 32
shoulder 33
dialyser end 34
tube connector 36
central portion 38
annulus 40
apertures 42
ramp 44
circumferential flange 46
arcuate flange 48
projection 50
annular recess 52
sliding clip 60
tube end 62
dialyser end 64
spline 66
depression 67
flexible wings 68
notch 69
pair of arms 70
cut-outs 72
cut-out 74
hook 76
projection 78
chamfer 79
O-ring seal 90
dialyser filter connector port 110
circumferential recess 112
dialysate inlet port 114
dialysate outlet port 116
blood inlet port 122
arterial blood line 124
blood outlet port 126
venous blood line 128
disposable cartridge 200
clean dialysate outlet port 202
spent dialysis inlet port 204
hinged latch 302
end 304
wedge shape 308
projection 310
ledge 312
recess 314
distance D
gap G

1. A dialyser connector comprising:
a main body, and
a sliding clip,
wherein:
the main body includes a tube end and a dialyser end,
the main body defines a cavity, at least one aperture and at least one ramp, and
the sliding clip has a tube end and a dialyser end,
the sliding clip defines a spine connecting the tube end and a dialyser end,
the sliding clip includes at least one flexible wing extending in an arc from the spine and at least one arm extending in an arc from the spine,
the at least one arm includes at least one projection extending radially inward,
the dialyser connector includes a locked condition in which the at least one projection extends into the cavity of the main body and an unlocked conditions in which the at least one projection is withdrawn from the cavity of the main body,
and
the at least one projection is biased to the locked condition by resilient abutment of the at least one flexible wing of the at lease one ramp.

2. A dialyser connector according to claim 1, wherein the at least one projection comprises a pair of projections and wherein the at least one aperture comprises a pair of apertures.

3. A dialyser connector according to claim 1, wherein the at least one arm includes a hook configured to abut a shoulder of the main body to prevent detachment of the sliding clip from the main body when assembled.

4. A dialyser connector according to claim 1, wherein the central portion is bounded toward the tube end by a circumferential flange, preferably wherein the central portion is partially bounded toward the dialyser end by at least one arcuate flange.

5. A dialyser connector according to claim 1, wherein the spine includes a depression.

6. A dialyser connector according to claim 1, wherein the main body includes a projection, preferably wherein the spine includes a notch.

7. A dialyser connector according to claim 6, wherein the projection is aligned with the vertical plane and extends from an upper surface of the main body.

8. A dialyser connector according to claim 1, further comprising a seal, preferably wherein the seal comprises an O-ring seal.

9. A dialyser connector according to claim 8, wherein the seal comprises an overmould on the main body.

10. A dialyser connector according to claim 1, wherein the dialyser connector is symmetrical about a centered vertical plane.

11. A dialyser connector according to claim 1, wherein the dialyser connector is single use, and comprises plastic.

12. A dialyser connector according to claim 1, wherein the at least one arm includes a hinged latch.

13. A dialyser connector according to claim 12, wherein the hinged latch is connected to the at least one arm by a living hinge.

14. A dialyser connector according to claim 12, wherein the hinged latch terminates in a wedge shape and the at least one arm comprises a pair of arms such that the other of the pair of arms includes a corresponding ledge.

15. A dialysate mixing and pumping cartridge comprising a dialyser connector as defined in claim 1.

16. A method of assembling a dialyser connector according to claim 1 comprising the steps of:
inserting an O-ring seal axially into the main body from the dialyser end;
mounting the sliding clip over the main body; and
slotting the at least one arm over the central portion with the at least one projection extending into the at least one aperture with a snap fit connection, wherein the at least one arm comprises a hook which abuts a shoulder of the main body to prevent detachment of the sliding clip from the main body when assembled.

17. A method of connecting a dialyser connector to a dialyser filter connector port, the method comprising the steps of:
providing a dialyser connector as defined in claim 1,
depressing the spine of the sliding clip relative to the main body so as to withdraw the at least one projection from the recess of a dialyser filter connector port,
connecting the dialyser connector to the dialyser filter connector port,
and
releasing the spine of the sliding clip relative to the main body so that the dialyser connector automatically assumes a locked condition in which the at least one projection engages a recess of a dialyser filter connector port under the biasing action of the at least one wing on the at least one ramp.

18. A method according to claim 17, further comprising the step of disconnecting the dialyser connector from the dialyser filter connector port by:
depressing the spine of the sliding clip relative to the main body so as to withdraw the at least one projection from the recess of a dialyser filter connector port,
and
withdrawing the dialyser connector from the dialyser filter connector port.
